(19) United States
(12) Patent Application Publication
(10) Pub. No.: US 2026/0215411 A1
(43) Pub. Date: Jul. 30, 2026

(54) HYPERBARIC OXYGEN CONTAINER

(71) Applicant: Ali SAHAN, Ankara (TR)

(72) Inventor: Ali SAHAN, Ankara (TR)

(21) Appl. No.: 19/150,195

(22) PCT Filed: Dec. 29, 2023
(86) PCT No.: PCT/TR2023/051865

§ 371 Date: Jul. 23, 2025

(30) Foreign Application Priority Data
Jan. 24, 2023 (TR) 2023/000853

(51) Int. Cl. A01N 1/145; A61B 17/00

(52) U.S. Cl. CPC A01N 1/145; A61B 17/00; A61B 2017/00752

(57) ABSTRACT

A hyperbaric oxygen container includes a housing, a lid sealing the housing and removably mounted on the housing, a cell preservation plate suitable for being placed into the housing, a pressure inlet means located on the housing and allowing the pressure in the housing to be increased and a pressure relief means located on the housing and allowing the pressure in the housing to be reduced.

The present invention relates to a hyperbaric oxygen container which ensures that the structures that should remain viable such as organs, tissues, follicles stored in the container are oxygenated and kept under appropriate temperature and pressure, and to a hair transplantation method.

Hyperbaric oxygen treatment, which ensures that the body receives the oxygen it needs to heal itself and is known as high-pressure oxygen treatment, has been practiced since the seventeenth century. In hyperbaric oxygen treatment centers of hospitals, hyperbaric oxygen treatments, which are practiced in many health problems such as carbon monoxide intoxication, diabetes, wound treatments, and decompression sickness, are based on the principle that individuals enter a tubular chamber and receive pure oxygen. Hyperbaric oxygen treatment is a treatment that increases the oxidation of tissues by creating more pressure than normal atmospheric pressure. Owing to the pressurized oxygen applied, the amount of oxygen dissolved in the liquid part of the blood increases substantially and the oxygen content in the tissues is maximized. In this treatment, which is applied in a cabin where patients enter and receive treatment, the whole body is usually exposed to this procedure.

In hair transplantation procedures that have become popular recently, hyperbaric oxygen treatment methods can be used to promote hair growth. In known methods, the scalp is usually placed in a closed environment and exposed to intense oxygen gas. However, when hair follicles or tissues are extracted from the human body and transplanted back into different tissues, their oxygenation and nutrition are disrupted in the elapsed time. During this period when there is no oxygenation, the hair follicle or tissue product that is damaged during transfer from one part of the body to another part or person loses its viability, and therefore the desired effect cannot be achieved.

Chinese patent document no CN114642538, known in the state of the art, discloses a device for promoting hair growth. The helmet-shaped device is placed on the scalp and oxygen is supplied to the hair follicles under certain pressure values.

The object of the invention is to provide a hyperbaric oxygen container and a cosmetic hair transplantation method that prevent the permanent hair loss rates after transplantation in hair transplantation procedures.

Another object of the invention is to provide a hyperbaric oxygen container that is portable and allows the placement of living tissue therein.

The figures do not limit the scope of protection defined in the claims and they should not be used alone for the purpose of interpreting the scope defined in the claims without reference to the technical description in the description of the present invention.

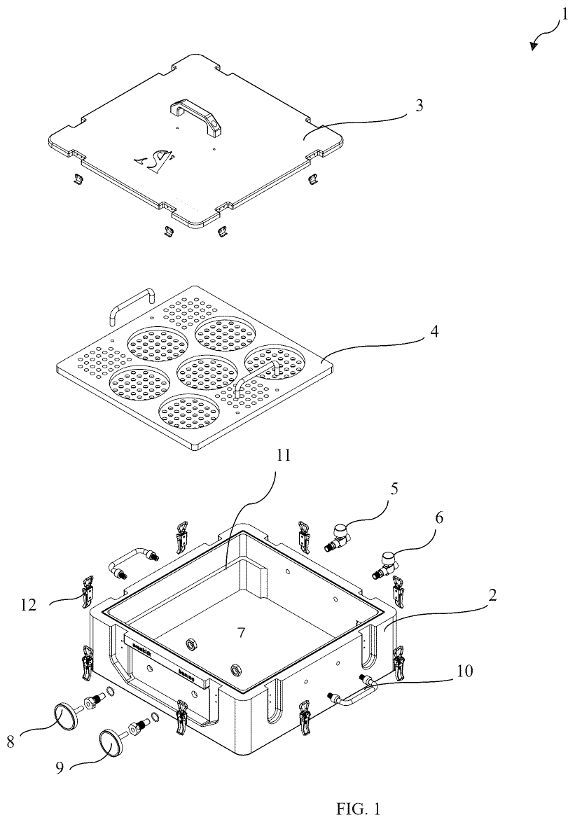

FIG. 1—A perspective view of a hyperbaric oxygen container in an embodiment of the invention.

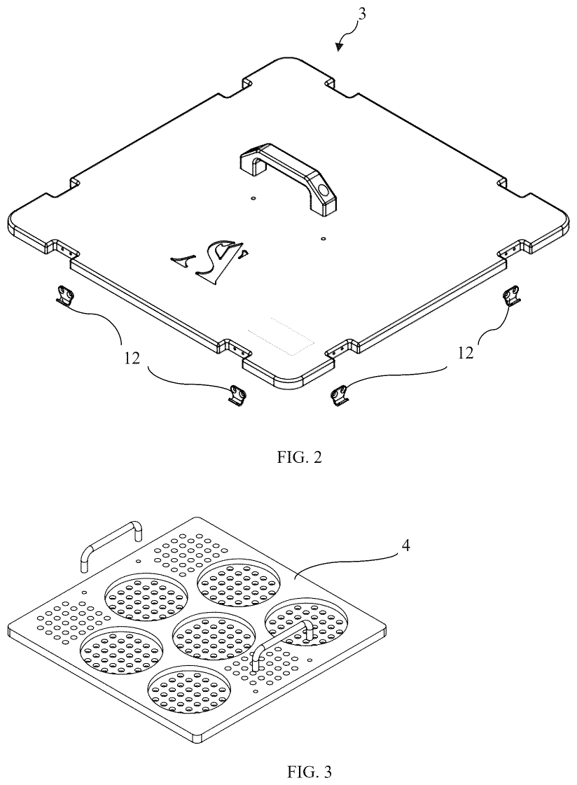

FIG. 2—A perspective view of a lid in an embodiment of the invention.

FIG. 3—A perspective view of a cell preservation plate in an embodiment of the invention.

The components shown in the figures are each given reference numbers as follows:
1. Hyperbaric oxygen container
2. Housing
3. Lid
4. Cell preservation plate
5. Pressure inlet means
6. Pressure relief means
7. Temperature control system
8. Thermometer
9. Barometer
10. Transfer means
11. Support element
12. Fastening element

The hyperbaric oxygen container (1) comprises a housing (2), a lid (3) sealing the housing (2) and removably mounted on the housing (2), a cell preservation plate (4) suitable for being placed into the housing (2), a pressure inlet means (5) located on the housing (2) and allowing the pressure in the housing (2) to be increased, and a pressure relief means (6) located on the housing (2) and allowing the pressure in the housing (2) to be reduced. After the cell preservation plate (4), in which the hair follicle or tissue product is placed, placed in the housing (2), the lid (3) is mounted on the housing (2) in such a way as to prevent any gas pass. Then, oxygen is allowed to enter the housing (2) by means of an oxygen cylinder through the pressure inlet means (5). After providing a pressure of approximately 2 atm in the hyperbaric oxygen container (1), the oxygen cylinder is separated. After use, the pressure in the hyperbaric oxygen container (1) must be brought to 0 atm when the lid (3) is opened or in the transfer position. This procedure is performed by means of the pressure relief means (6).

The hyperbaric oxygen container (1) according to the invention comprises a temperature control system (7) located in the housing (2), which ensures that the ambient temperature is kept within a predetermined temperature range. By means of the temperature control system (7), the oxygen content of the air in the hyperbaric oxygen container (1) is kept at an optimum level, and hair follicles and tissues are ensured to maintain their viability effectively. Exposing the hair follicle or tissue products to be transplanted into the scalp to high-pressure oxygen at a temperature range predetermined by the manufacturer prevents undesirable conditions such as ischemia or necrosis.

In one embodiment of the invention, the hyperbaric oxygen container (1) comprises a thermometer (8) placed on the housing (2). In this way, the temperature changes of the cell preservation plate (4) can be precisely monitored.

In one embodiment of the invention, the hyperbaric oxygen container (1) comprises a barometer (9) placed on the housing (2). In this way, the pressure to which the cell preservation plate (4) is exposed can be monitored and emergency action to the hyperbaric oxygen container (1) can be performed.

In one embodiment of the invention, the hyperbaric oxygen container (1) comprises at least one transfer means (10) located on the housing (2) and allowing the housing (2) to be transferred. By this means, the hyperbaric oxygen container (1) can be transferred easily. In one embodiment of the present invention, transfer means (10) is grip-shaped, and the grips are located on both sides of the housing (2). In a further embodiment of the present invention, the grips are foldable, therefore after the transfer process, the hyperbaric oxygen container (1) takes up less space when in use or storage.

In one embodiment of the invention, the hyperbaric oxygen container (1) comprises a support element (11) located in the housing (2) and suitable for the cell preservation plate (4) being placed thereon. In this way, the cell preservation plate (4) can be placed between the base of the housing (2) and the lid (3). If required by the user, the user can directly intervene in the ambient temperature with the cooling and heating elements to be placed under the cell preservation plate (4) during use.

In one embodiment of the invention, the hyperbaric oxygen container (1) comprises a temperature control system (7) which ensures that the temperature in the housing (2) is kept between 4 and 8° C. This temperature range is the organ storage temperature at which the metabolism of the cells is slowed down, excess energy expenditure is prevented, and the life of the organ is tried to be prolonged.

In one embodiment of the invention, the hyperbaric oxygen container (1) comprises at least one fastening element (12) located on the housing (2) and/or the lid (3) and allowing the housing (2) and the lid (3) to be hermetically fastened. In this embodiment of the invention, the fastening elements (12) are in the form of latches and locking is ensured with the help of latches after the lid (3) is closed on the housing (2).

In one embodiment of the invention, the inlet means (5) and/or the pressure relief means (6) are in the form of taps or valves. In this way, air transfer into and out of the hyperbaric oxygen container (1) can be provided in a bidirectional and controlled manner.

The hair transplantation method according to the invention comprises the steps of extracting the hair follicle from the scalp, providing oxygenation of the hair follicle in a hyperbaric oxygen container at a pressure of approximately 2 atm and a temperature range of 4 to 8° C., and transplanting the oxygenated hair follicle into the scalp. Owing to the cosmetic method, which comprises the steps of providing oxygenation of the hair follicles after extraction from the scalp in a hyperbaric oxygen container at a pressure of approximately 2 atm and a temperature range of 4 to 8° C., and transplanting the oxygenated hair follicle into the scalp, metabolism of the cells is slowed down, preventing them from excess energy expenditure and prolonging the life of hair follicles.

Thanks to the present invention, hyperbaric oxygen container (1) increases the oxygen level of the tissue or hair follicle placed in the cell preservation plate (4) with the effect of ambient oxygen pressure, and therefore the oxygen requirement of the product whose nutrition is disturbed, that is, oxygen deprived, is met and their survival time is prolonged.

What is claimed is:

1. A hyperbaric oxygen container suitable for hair follicle oxygenation, comprising:
a housing,
a lid sealing the housing and removably mounted on the housing,
a cell preservation plate suitable for being placed into the housing,
a pressure inlet means located on the housing and allowing pressure in the housing to be increased,
a pressure relief means located on the housing and allowing the pressure in the housing to be reduced, and
a temperature control system located in the housing and ensuring that an ambient temperature is kept within a predetermined temperature range.

2. The hyperbaric oxygen container according to claim 1, further comprising a thermometer placed on the housing.

3. The hyperbaric oxygen container according to claim 1, further comprising a barometer placed on the housing.

4. The hyperbaric oxygen container according to claim 1, further comprising at least one transfer means located on the housing and allowing the housing to be transferred.

5. The hyperbaric oxygen container according to claim 1, further comprising a support element located in the housing and suitable for the cell preservation plate being placed on the support element.

6. The hyperbaric oxygen container according to claim 1, further comprising the temperature control system ensuring that the ambient temperature in the housing is kept between 4° C. and 8° C.

7. The hyperbaric oxygen container according to claim 1, further comprising at least one fastening element located on the housing and/or the lid and allowing the housing and the lid to be hermetically fastened.

8. The hyperbaric oxygen container according to claim 1, wherein the pressure inlet means and/or the pressure relief means is in a form of taps or a form of valves.

9. A hair transplantation method, comprising the followings steps:
extracting a hair follicle from a scalp,
providing oxygenation of the hair follicle in a hyperbaric oxygen container at a pressure of 2 atm and a temperature range of 4° C. to 8° C. to obtain an oxygenated hair follicle, and
transplanting the oxygenated hair follicle into the scalp.

10. The hyperbaric oxygen container according to claim 2, further comprising a barometer placed on the housing.

11. The hyperbaric oxygen container according to claim 2, further comprising at least one transfer means located on the housing and allowing the housing to be transferred.

12. The hyperbaric oxygen container according to claim 3, further comprising at least one transfer means located on the housing and allowing the housing to be transferred.

13. The hyperbaric oxygen container according to claim 2, further comprising a support element located in the housing and suitable for the cell preservation plate being placed on the support element.

14. The hyperbaric oxygen container according to claim 3, further comprising a support element located in the housing and suitable for the cell preservation plate being placed on the support element.

15. The hyperbaric oxygen container according to claim 4, further comprising a support element located in the housing and suitable for the cell preservation plate being placed on the support element.

16. The hyperbaric oxygen container according to claim 2, further comprising the temperature control system ensuring that the ambient temperature in the housing is kept between 4° C. and 8° C.

17. The hyperbaric oxygen container according to claim 3, further comprising the temperature control system ensuring that the ambient temperature in the housing is kept between 4° C. and 8° C.

18. The hyperbaric oxygen container according to claim 4, further comprising the temperature control system ensuring that the ambient temperature in the housing is kept between 4° C. and 8° C.

19. The hyperbaric oxygen container according to claim 5, further comprising the temperature control system ensuring that the ambient temperature in the housing is kept between 4° C. and 8° C.

20. The hyperbaric oxygen container according to claim 2, further comprising at least one fastening element located on the housing and/or the lid and allowing the housing and the lid to be hermetically fastened.
